(19) United States
(12) Patent Application Publication
(10) Pub. No.: US 2022/0120706 A1
(43) Pub. Date: Apr. 21, 2022

(54) SENSING MATERIAL FOR HIGH SENSITIVITY AND SELECTIVITY

(71) Applicant: QI SENSOR TECHNOLOGIES LIMITED, HongKong (CN)

(72) Inventors: Han JIN, Ningbo, Zhejiang (CN); Wan Lung KAM, Hong Kong (CN); Wai Fung CHEUNG, Hong Kong (CN)

(21) Appl. No.: 17/434,419

(22) PCT Filed: Feb. 27, 2019
(86) PCT No.: PCT/CN2019/076229

§ 371 Date: Aug. 27, 2021

(51) Int. Cl. G01N 27/407; G01N 33/00

(52) U.S. Cl. CPC G01N 27/407; G01N 33/0036

(57) ABSTRACT

This invention provides a sensing electrode for detecting at least one target gas in a gas mixture having at least one interference gas. In one embodiment, the sensing electrode has: (a) a layer of sensing nanoparticles; (b) a reaction interface; and (c) a solid state electrolyte; each of the sensing nanoparticles has a catalytic core and a photoactive porous shell, the catalytic core breaks down said at least one interference gas, the photoactive porous shell enhances electrochemical reaction at said reaction interface when illuminated with light of a specific wavelength.

### CROSS-REFERENCE TO RELATED APPLICATIONS

Throughout this application, various publications are cited. The disclosures of these publications in their entireties are hereby incorporated by reference into this application to more fully describe the state of the art to which this invention pertains.

### FIELD OF THE INVENTION

This invention relates to the field of sensors.

### BACKGROUND OF THE INVENTION

At present, diagnosis of cancer often happens very late since subtle symptoms can be found for the cancer patients at early stage. More than half percentage of patients suffer from lung cancer at an advanced stage when they were told by doctor¹. Typically, less than 15% 5-years' survival rate is found for the patients at the advanced stage, while the 5-years' survival rate of stage I patients can even be higher than 88% by giving timely surgical treatment². Consequently, there will be a huge clinical demand for diagnosing cancer at a very early stage so that efficient clinical treatment can be provided to patients. Breath analysis has been widely considered as a non-invasive, safe and reliable way to observe the details of biological metabolic and physiological process in the human body. In the past few decades, numerous studies have shown that the smell of breath in patients is closely related to cancer^{3, 4}. Therefore, rapid and sensitive detection of volatile organic compounds (VOCs), namely the cancer volatile markers in breath samples, has the potential of early diagnosing cancer. Moreover, recent studies have demonstrated that specific trace volatile markers can be found for each tumor such as lung cancer, breast cancer, melanoma, colon cancer^{5, 6, 7}. With the utilization of VOCs tracking devices, the identification of lung cancer, breast cancer and colon cancer can be easily achieved by specifically sensing VOCs markers^{5, 6, 7}.

For early diagnosing cancer via a non-invasive way, monitoring the volatile markers with high sensitivity and specificity is one of the key scientific problems. Among various volatile markers tracking devices, portable sensors gain more attention owning to their low-cost, easy-to-use, require only low power for operation, and are inexpensive⁸. These gas sensors based on various metal oxides and/or functionalized noble metal nanoparticles have shown desirable sensing behavior in monitoring ppb (parts per billion) level VOCs⁹. However, one major problem with them is the inadequate identification capability when facing VOCs mixture. To date, the frequently reported strategy for addressing this remained challenging issue is to design an algorithm assisted sensor array^{10, 11, 12, 13}. For instance, a light-regulated electrochemical sensor array has been developed with acceptable identification feature and enhanced sensitivity for detecting 6 kinds of VOCs although complex data processing algorithm is required¹⁴. Beyond designing sensor array, searching advanced materials provides an alternative strategy to improve the sensing properties. Quite recently, Jong-Heun Lee et. al. announced nanoscale TiO₂ or SnO₂ catalytic overlayer can effectively remove interference gases and achieved remarkable selectivity to specific gases¹⁵. Nevertheless, the catalytic layer also reduced the amount of target gases reaching the reaction sites when filtering interference gases, resulting in the relatively low response signal.

It was previously proposed the light-regulated electrochemical reaction which can significantly enhance the response signal and sensitivity as well as low detection limit¹⁶. It is speculated that if the light-regulated reaction can be combined with the catalytic overlayer, there will be the possibility of obtaining satisfactory response behavior, namely high sensitivity and selectivity, in monitoring volatile markers. Theoretically, core-shell sensing materials with porous shell and catalytic core can selectively remove uninterested gases, since gas mixture can easily reach the catalytic core by diffusing through porous shell. If a photoactive shell is used, then the light-regulated electrochemical reaction can be triggered when been illuminated, leading to the high sensitivity and satisfactory selectivity. In other words, the photoactive shell will be designed for trigging the light-regulated electrochemical reaction to enhance the response magnitude while catalytic active core will play the function of removing interference gases. Based on this assumption, the practicability of designing a light-regulated electrochemical reaction assisted core-shell structure will be confirmed in the present invention. Impact of the species for the catalytic core used in this invention and the shell thickness on the response behavior will be explored and discussed to enrich understanding of artificially tailoring the sensitivity and selectivity of the sensor, particular, to provide an alternative approach designing high-performance VOCs tracking devices for future clinic use.

### SUMMARY OF THE INVENTION

This invention provides a sensing electrode for detecting at least one target gas in a gas mixture having at least one interference gas. In one embodiment, said sensing electrode comprises: (a) a layer of sensing nanoparticles; (b) a reaction interface; and (c) a solid state electrolyte; wherein each of said sensing nanoparticles comprises a catalytic core and a photoactive porous shell, said catalytic core breaks down said at least one interference gas, said photoactive porous shell enhances electrochemical reaction at said reaction interface when illuminated with light of a specific wavelength.

This invention further provides a sensor comprising said sensing electrode and a method for detecting at least one target gas in a gas mixture having at least one interference gas using said sensing electrode. In one embodiment, said method comprises the steps of (a) providing said sensing electrode and a reference electrode; (b) illuminating said sensing electrode with light of said specific wavelength; (c) providing said gas mixture to said sensing electrode; and (d) measuring electric potential difference between said sensing electrode and said reference electrode.

### BRIEF DESCRIPTION OF THE DRAWINGS

FIG. 1. Illustration of the overall experimental strategy: (a) inadequate sensitivity and poor selectivity is generally found for the electrochemical gas sensors (e.g. yttria-stabilized zirconia-based sensors); (b) schema of the core-shell sensing materials with porous photoactive shell and catalytic active core; (c) due to the filter effect, interference gases can be removed by the catalytic active core. However, the level of the target may also be partly reduced, leading to the low sensitivity and high selectivity of the sensor when operated at light off (without illumination); (d) after been illuminated, response signal of the sensor can be significantly enhanced. Herein, satisfactory sensitivity and selectivity as well as the low detection limit are expected.

FIG. 2. Schema of the sensing behavior for the electrochemical sensor that exposed to the VOCs mixture.

FIG. 3. Schema of the sensing behavior for the electrochemical sensor comprised of the core-shell sensing electrode.

FIG. 4. Illustration of the sensing performance for the electrochemical sensor using photoactive sensing materials, operated at (a) light off; (b) light on.

FIG. 5. Conversion rate of various volatile markers at 425° C., catalyzed by various metal oxides or noble metal.

FIG. 6. XRD patterns of the as-synthesized ZnO, Fe₂O₃ and Fe₂O₃@ZnO (derived from different amount of zinc acetate precursor).

FIG. 7. HRTEM images of (a) shuttle-like Fe₂O₃; (b) ZnO; (c) Fe₂O₃@ZnO derived from 0.05 mol/L; (d) 0.15 mol/L; (e) 0.25 mol/L and (f) 0.35 mol/L zinc acetate precursor. Fe₂O₃@ZnO core-shell heterostructure is successfully synthesized and extra ZnO particles can be found when the amount of zinc acetate precursor is higher than 0.25 mol/L.

FIG. 8. EDX analysis of Fe₂O₃@ZnO core-shell heterostructure that derived from (a) 0.05 mol/L; (b) 0.15 mol/L; (c) 0.25 mol/L and (d) 0.35 mol/L zinc acetate precursor.

FIG. 9. Schema for the impact of the shell thickness on the sensing performance of the sensor. (a) Thick shell blocks the filter effect while (b) extremely thin shell may not be able to trigger the light-regulated electrochemical reaction. It is expected that the electrochemical sensor comprised of core-shell heterostructure with modest shell thickness could demonstrate desirable sensing behavior.

FIG. 10. HRTEM images of the (a) shuttle-like Fe₂O₃; (b)-(d) Fe₂O₃@ZnO core-shell heterostructure derived from different amount of zinc acetate precursor. Low/high level of zinc acetate precursor features the Fe₂O₃@ZnO with extremely thin/thick shell while modest shell thickness is formed after adding modest amount of zinc acetate precursor.

FIG. 11. (a) Response patterns for the electrochemical sensor comprised of Fe₂O₃—, ZnO— or Fe₂O₃@ZnO (with diverse shell thickness)-SE (vs. Mn-based RE), depicted in the form of a heat map; (b) response magnitude for the electrochemical sensor using Fe₂O₃@ZnO (with shell thickness of 4.8 nm)-SE vs. Mn-based RE, operated at light off or on; (c) dependence of the response signal (ΔV) on the logarithm of 3-methylhexane concentration in the range of 0.8-5 ppm; (d) humidity effects on the response magnitude of the sensor operated at light off and on; (e) long-term stability of the sensor to 5 ppm 3-methylhexane within 14 days, operated at light on. It can be seen that Fe₂O₃@ZnO (with shell thickness of 4.8 nm) offers the sensor acceptable selectivity to 3-methylhexane. In particular, sensing properties of the sensor is significantly enhanced by illumination. Water vapor gives minor impact on the sensing performance of the sensor regardless of operating at light off or on. Moreover, acceptable stability in the response behavior is confirmed for the sensor within 14 days continuously measurement.

FIG. 12. Sensing properties of the electrochemical sensor (fabricated at different sintering temperature) to 6 kinds of VOCs, comprised of the Fe₂O₃@ZnO (with shell thickness of 4.8 nm)-SE vs. Mn-based RE.

FIG. 13. Variation of the response magnitude and 90% response/recovery time of the electrochemical sensor (using Fe₂O₃@ZnO-SE, with shell thickness of 4.8 nm) to 5 ppm 3-methylhexane at different operating temperature.

### DETAILED DESCRIPTION OF THE INVENTION

Breath analysis has been considered as a non-invasive, safe and reliable way to diagnose cancer at a very early stage. Rapid detection of cancer volatile markers in breath samples via a portable sensing device will lay the foundation of future early cancer diagnosis. Nevertheless, unsatisfactory sensitivity and specificity of these sensing devices restrain the clinical application of breath analysis. Herein, it is proposed the strategy of designing the light-regulated electrochemical reaction assisted core-shell heterostructure to address the concerned issue, namely, the photoactive shell will be designed for trigging the light-regulated electrochemical reaction and enhancing the sensitivity while catalytic active core will play the function of removing interference gases. After screening of various core candidates, Fe₂O₃ was found to exhibit a relatively low conversion rate to 3-methylhexane, suggesting the mutual interference would be eliminated by Fe₂O₃. Based on the assumption, the electrochemical sensor comprised of core-shell Fe₂O₃@ZnO-SE (vs. Mn-based RE) was fabricated and sensing properties to 6 kinds of volatile markers were evaluated. Interestingly, the thickness of ZnO shell significantly influenced the response behavior, typically, the Fe₂O₃@ZnO with the shell thickness of 4.8 nm offers the sensor high selectivity to 3-methylhexane. In contrast, significantly mutual response interference is observed for the Fe₂O@ZnO with an extremely thick/thin shell. Particularly, sensing properties are greatly enhanced upon illumination, detection limit to 3-methylhexane can even down to 0.072 ppm which will be useful in clinic application. In summary, the strategy proposed in this invention is expected to be a starting point for artificially tailoring the selectivity of future sensing devices.

In one embodiment, this invention provides a sensing electrode for detecting at least one target gas in a gas mixture having at least one interference gas, said sensing electrode comprises: (a) a layer of sensing nanoparticles; (b) a reaction interface; and (c) a solid state electrolyte; wherein each of said sensing nanoparticles comprises a catalytic core and a photoactive porous shell, said catalytic core breaks down said at least one interference gas, said photoactive porous shell enhances electrochemical reaction at said reaction interface when illuminated with light of a specific wavelength.

In one embodiment, said photoactive porous shell has a thickness of 3 nm to 10 nm e.g. 3.9 nm, 4.8 nm, 5.2 nm or 7.5 nm. In another embodiment, said photoactive porous shell has a thickness of 4 nm to 6 nm. In a further embodiment, said photoactive porous shell has a thickness of 3, 3.5, 4, 4.5, 5, 5.5, 6, 6.5, 7, 7.5, 8, 8.5, 9, 9.5 or 10 nm.

In one embodiment, said catalytic core has an average size of 150 nm to 400 nm e.g. 198 nm, 234 nm or 264 nm. In another embodiment, said catalytic core has an average size of 150, 200, 250, 300, 350 or 400 nm.

In one embodiment, said catalytic core has a shuttle-like morphology. In another embodiment, said catalytic core has spherical morphology or any other morphologies.

In one embodiment, said catalytic core is a metal oxide or metallic nanoparticle. In another embodiment, said metal oxide or metallic nanoparticle is selected from the group consisting of Fe₂O₃, In₂O₃, Au, Ag and Nb₂O₅

In one embodiment, said photoactive porous shell is made of ZnO. In another embodiment, said photoactive porous shell is ZnO based materials. In a further embodiment, said ZnO based materials is selected from the group consisting of ZnO+x % In₂O₃, wherein x≤40, e.g. 5, 10, 15, 20, 25, 30, 35 or 40.

In one embodiment, said target gas comprises a 3-methyl-alkyl group. In another embodiment, said target gas is 3-methylhexane.

In the same embodiment, said interference gas selected from the group consisting of benzene, styrene, nonane, hexane, 3-methylhexane, 2-ethylhexanol, 3-methylhexane, 5-ethyl-3-methyloctane, acetone, ethanol, ethyl acetate, ethyl-benzene, isononane, isoprene, nonanal, styrene, toluene, and undecane.

In one embodiment, said specific wavelength ranges from 360-840 nm. In another embodiment, said specific wavelength ranges from 380-840 nm.

In one embodiment, said solid state electrolyte is an oxygen ion conductor. In another embodiment, said solid state electrolyte is yttria-stabilized zirconia.

In one embodiment, wherein said catalytic core breaks down said at least one interference gas at a temperature above 400° C. In another embodiment, said catalytic core breaks down said at least one interference gas at a temperature 400-470° C.

In one embodiment, a sensor comprising said sensing electrode is provided by this invention.

In one embodiment, a method for detecting at least one target gas in a gas mixture having at least one interference gas using the sensing electrode of this invention is provided. In one embodiment, said method comprises the steps of: (a) providing said sensing electrode and a reference electrode; (b) illuminating said sensing electrode with light of said specific wavelength; (c) providing said gas mixture to said sensing electrode; and (d) measuring electric potential difference between said sensing electrode and said reference electrode.

In one embodiment, said step (c) is conducted at a temperature above 400° C. In another embodiment, said step (c) is conducted at a temperature 400-470° C.

In one embodiment, said target gas is at a concentration of 0-100 ppm. In another embodiment, said target gas is at a concentration of 0.07-5 ppm.

In one embodiment, said interference gas is at a concentration below 5 ppm. In one embodiment, said interference gas is at a concentration of 0.8-5 ppm.

In one embodiment, said target gas comprises a 3-methyl-alkyl group.

In one embodiment, said interference gas is selected from the group consisting of benzene, styrene, nonane, hexane, 3-methylhexane, 2-ethylhexanol, 3-methylhexane, 5-ethyl-3-methyloctane, acetone, ethanol, ethyl acetate, ethyl-benzene, isononane, isoprene, nonanal, styrene, toluene, and undecane.

### EXAMPLES

### Screen of Core Candidates:

The conversion rate of the selected core candidates to 6 kinds of reported representative volatile markers (benzene, styrene, 3-methylhexane, nonane, hexane and acetone) is carried out with the similar way mentioned previously¹⁷. In brief, 100 ppm specific VOC (diluted with air base) was flowed through 15 mg various core candidates powder at 425° C. with the rate of 100 mL/min. Changes in VOC concentration in the gas outlet were measured via the gas chromatography (GC, GC-6890A, Zhongkehuifen, China) to obtain the converted percentage.

### Synthesis of Sensing Materials and Materials Characterization:

Details of the synthesizing route for the Fe₂O₃, ZnO and Fe₂O₃@ZnO core-shell sensing material can be found elsewhere¹⁸. The crystal phase, microstructure and elemental analysis of the sensing materials were characterized by means of the X-ray.

Diffractometor (XRD; Rigaku Ultima IV. Japan), field-emission scanning electron microscope (FESEM, SU-70, Hitachi, Japan) and high-resolution transmission electron microscope (HRTEM; FEI Tecnai G2 f20 s-twin) operated at 200 kV with the energy dispersive X-ray (EDS) analysis.

### Electrochemical Sensor Fabrication and Evaluating the Sensing Properties:

In fabrication of the electrochemical sensors, all the sensing materials were thoroughly mixed with α-terpineol and individually painted on the surface of yttria-stabilized zirconia (YSZ) plates (length×width×thickness: 2×1×0.2 cm; Nikkato, Japan) to form the 4 mm sensing layer. After drying over night, YSZ plates were sintered at high temperature in the range of 800-1000° C. (with intervals of 50° C.) to form the sensing electrode (SE). In order to simplify the sensor configuration, Mn-based reference electrode (RE) was used in the sensor¹⁷, fabricated with a similar way.

Both SEs and Mn-based RE of the sensor are simultaneously exposed to the base gas (diluted with air base) or the sample gas containing each of various VOCs (benzene, styrene, 3-methylhexane, nonane, hexane and acetone) to evaluate the gas sensing characteristics. Since a pre-concentrator is frequently used for the VOCs tracking devices to concentrate the VOCs (at ppb level) to several ppm when monitoring the VOCs exhaled from human breath, all the sample gases in the range of 1-5 ppm are selected. Initially, the sensor is operated without illumination (light off) and the sensing performance is recorded. Then, the sensing behavior of the sensor is examined by exposure to illumination (light on). Finally, the electric potential difference (ΔV, ΔV=V_{sample gas}−V_{base gas}) between SE and RE is recorded by using an electrometer (34970A, Agilent, USA). The distance between the sensor and LED lamp (Juhong, China, 17 μW/cm², 380-840 nm) is about 10 cm and the operating temperature is ranged from 400-475° C. The detection limit of the sensor is extrapolated at a signal-to-noise ratio of 3. The background relative humidity (R.H.) of the carrier gas was regulated by careful mixing of dry and fully humidified air (R. H. 100%). The ratio of the partial pressure of water vapor to the equilibrium vapor pressure of water at the same temperature, in the mixture was monitored by a hygrometer (4185 Traceable, USA) at room temperature (25-27° C.).

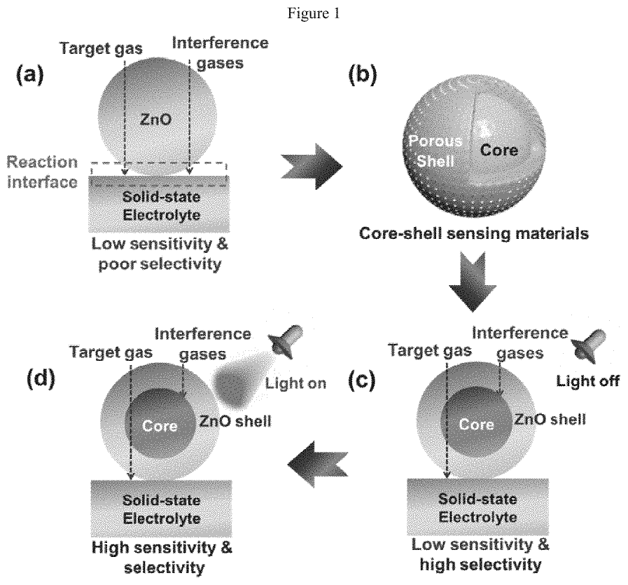

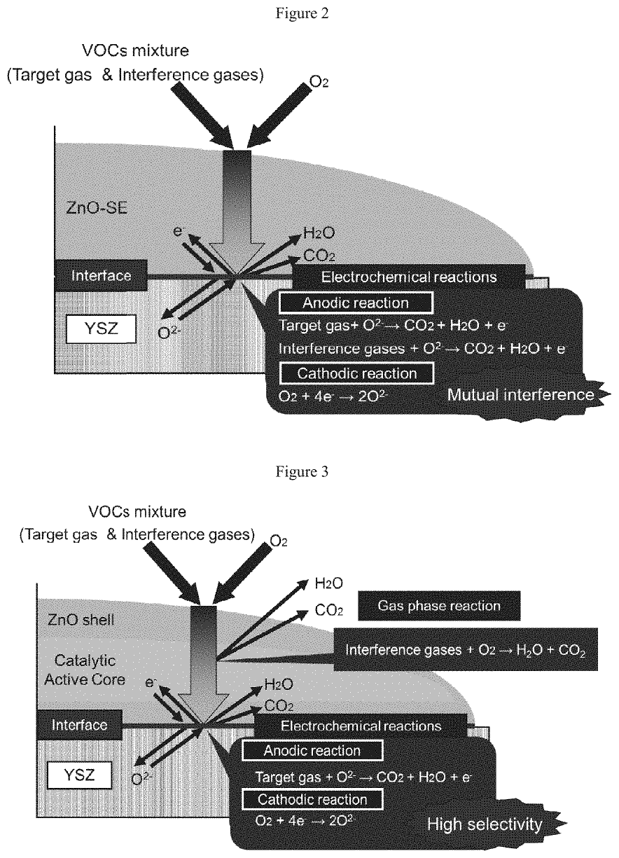

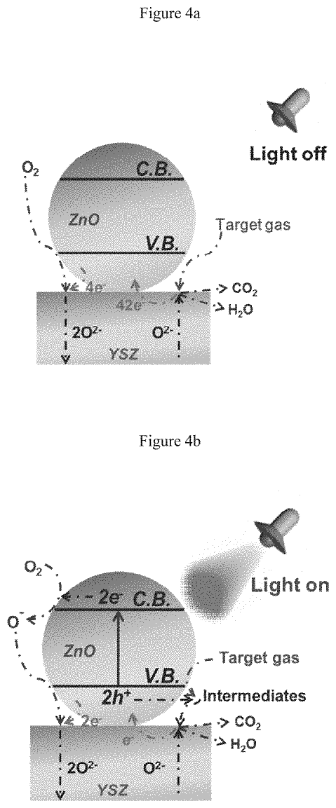

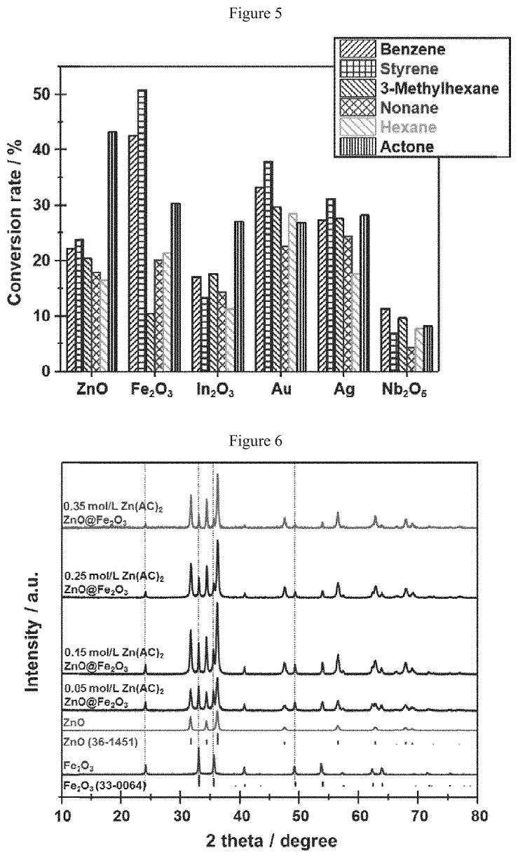

When electrochemical sensors exposed to the gas mixture, response signal to target gases and interference gases will be simultaneously generated. Since, minor difference in the electrocatalytic activity of the sensing materials (e.g. ZnO) can be found to target gas and interference gases, significantly mutual interference occurred (FIG. 1(a) and FIG. 2). When a porous core-shell sensing material is used in which the core can selectively remove interference gases, response signal to target gas will be solely generated by the sensor (FIG. 3). Note that part of target gas will be possibly converted before reaching the reaction interference, hence, relatively low response signal will be given by the electrochemical sensor (as shown in FIG. 1(b), (c)). Nevertheless, if a photoactive (e.g. ZnO) is coated on the surface of core and is illuminated (FIG. 1(d) and FIG. 4), satisfactory sensing behavior is expected since response signal to the target gas can be enhanced by the illumination although gas concentration participated the electrochemical reaction is partly reduced by the catalytic active core, namely, electrochemical sensor comprised of a porous & photoactive core-shell sensing material may simultaneously offer high sensitivity and selectivity as well as the low detection limited.

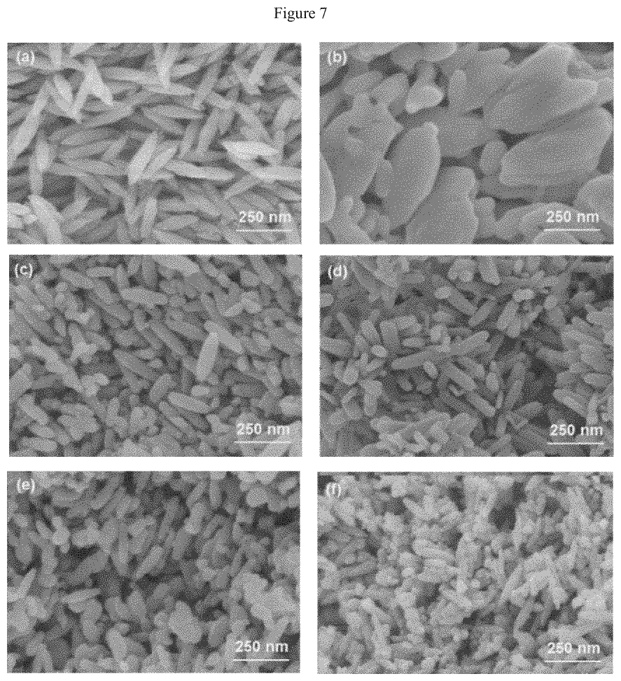

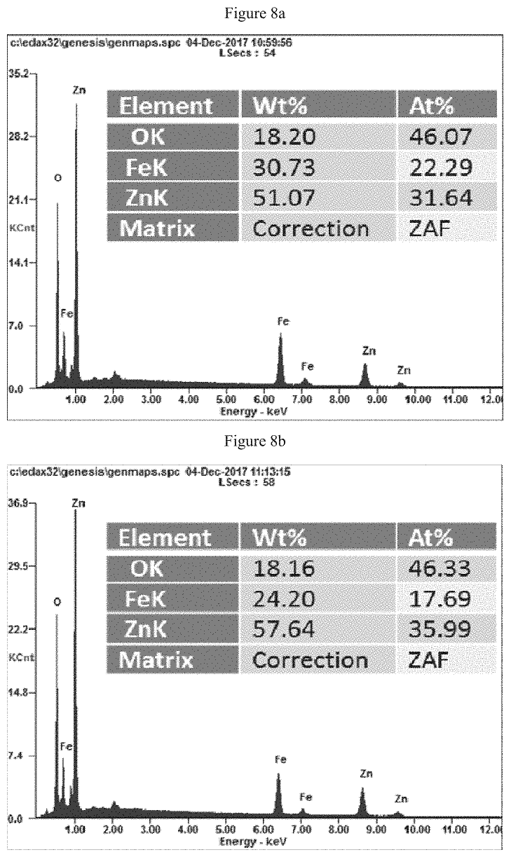

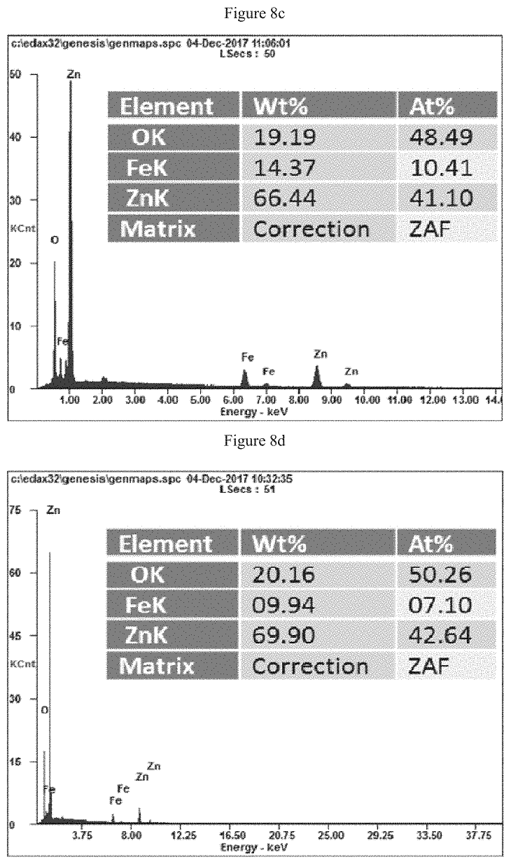

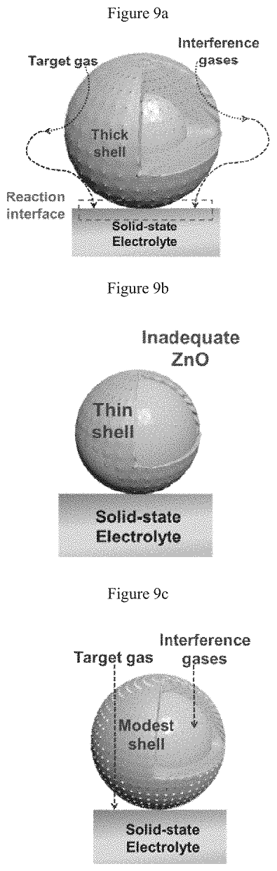

For the purpose of efficiently removing interference gases, metal oxides or metallic particles, e.g. Fe₂O₃¹⁸, In₂O₃¹⁹, Au²⁰, Ag²¹ and Nb₂O₅²² that can form core-shell heterostructure with photoactive ZnO shell are selected as the core candidates and their conversion rate to 6 kinds of reported representative volatile markers⁶ (benzene, styrene, 3-methylhexane, nonane, hexane and acetone) have been examined. The related details can be found in FIG. 5. Briefly, Fe₂O₃ demonstrates an obviously low conversion rate to 3-methylhexane while analogous conversion rate to all these studied VOCs is witnessed for other studied candidates. This important information suggests that the mutual interference would be eliminated by Fe₂O₃, offering the electrochemical sensor acceptable selectivity to 3-methylhexane. To confirm this assumption, Fe₂O₃@ZnO core-shell heterostructures with different shell thickness are synthesized via the generally reported hydrothermal route, the thickness of the ZnO shell is adjusted by adding a different amount of zinc acetate precursor. FIG. 6 shows the X-ray diffraction (XRD) patterns for the sample of core-shell Fe₂O₃@ZnO (with different shell thickness). For comparison, the XRD patterns of Fe₂O₃ and ZnO synthesized with the above mentioned approach¹⁸ are also included. As can be seen in FIG. 6 that the as-synthesized Fe₂O₃ and ZnO belong to the pure hematite (JCPDS No. 33-0064) and zincite (JCPDS No. 36-1451) phase. After coating with ZnO shell, the diffraction intensity of Fe₂O₃ peaks at 24.138 degree, 33.152 degree, 35.611 degree and 49.479 degree decreased with the increase in the amount of zinc acetate precursor, indicating the Fe₂O₃@ZnO core-shell heterostructure may be synthesized. Besides, the decreased diffraction intensity of Fe₂O₃ peak indirectly indicates the thickness of the ZnO shell may be adjusted by the amount of zinc acetate precursor. However, it must be particularly noted that the Fe₂O₃/ZnO powder mixture may also exist in the as-synthesized Fe₂O₃@ZnO core-shell sample. In order to confirm the success of synthesizing core-shell heterostructure, the microstructure of the obtained sample is further investigated by the FESEM (FIG. 7) and the relevant element contents are also analyzed by EDX mapping (FIG. 8). The high-magnification FESEM images in FIG. 6S reveal that after adding zinc acetate precursor, rough surface of Fe₂O₃ (with an average diameter of around 234 nm) becomes smoother (FIG. 7 (a-d)). This implies the ZnO shell was successfully coated on the surface of shuttle-like Fe₂O₃. Additionally, it can be seen that when the amount of zinc acetate precursor is higher than the value of 0.25 mol/L, ZnO particles start to appear and gradually form the ZnO/Fe₂O₃@ZnO powder mixture (FIG. 7(e)), in particular, for the sample derived from 0.35 mol/L zinc acetate precursor. Since, the extra existence of ZnO particles contributes minor effects to the selectivity and sensitivity, it is better to confine the level of zinc acetate precursor within 0.25 mol/L. The success of obtaining Fe₂O₃@ZnO core-shell heterostructurc is also proved by the EDX mapping images (FIG. 8). By increasing the zinc acetate content, the elemental fraction of Fe in the sample declined, particularly, the elemental of Zn dominated the whole sample derived from 0.35 mol/L zinc acetate precursor. This is well matched with the results shown in FIG. 6S.

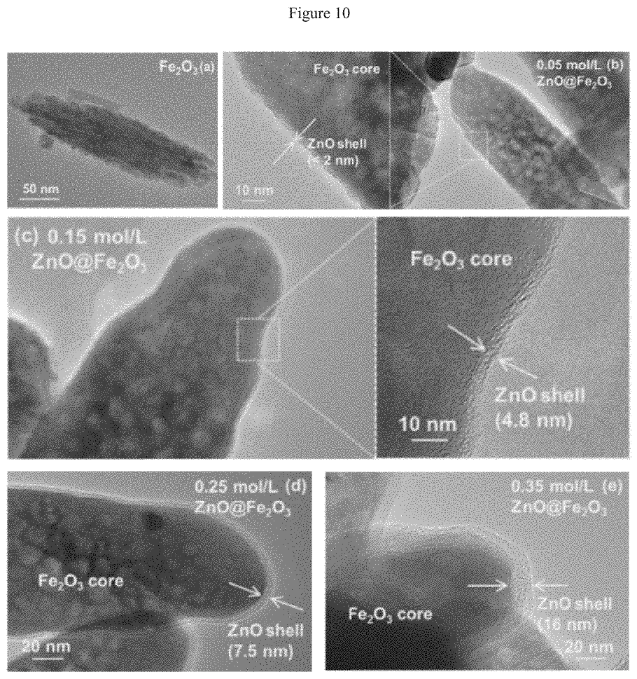

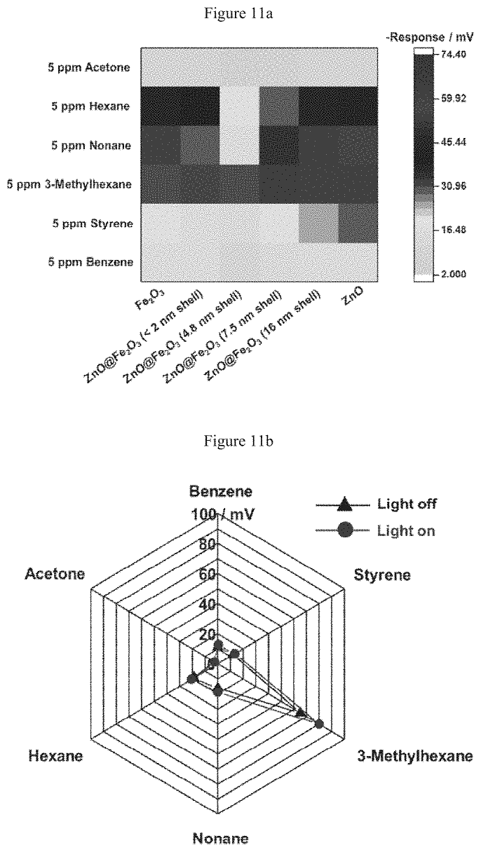

Beyond the species of the core candidate, thickness of the obtained Fe₂O₃@ZnO core-shell samples is another concerned parameter. Typically, a thick shell blocks the gas diffusion, leading to the interference gas can't be efficiently removed by the catalytic active core since filter effect is physically inaccessible (as shown in FIG. 9(a)). On the contrary, Fe₂O₃@ZnO with an extremely thin shell may not be able to trigger the light-regulated reaction owing to the inadequate ZnO on the surface. Besides, an extremely thin shell may result in the Fe₂O₃ directly contact the electrolyte (YSZ in this research), the electrochemical reaction will be generated by both Fe₂O₃ and ZnO-SEs. In this case, extra mutual interference will also be observed (FIG. 9(b)). Consequently, Fe₂O₃@ZnO with the tailor-made shell thickness is ultra-important for reaching the main research objective of the study (FIG. 9(c)). In order to have a clear vision of the influence of the zinc acetate content, HRTEM is taken of these samples and the corresponding images are shown in FIG. 10. In brief, the amount of zinc acetate precursor participated in the hydrothermal reaction significantly influences the ZnO shell thickness. Thick shell (around 16 nm) is formed after adding 0.35 mol/L zinc acetate while ZnO shell (less than 2 nm) can be hardly seen after adding 0.05 mol/L zinc acetate. In addition, Fe₂O₃@ZnO with modest shell thickness can be witnessed at the zinc acetate content of 0.15 mol/L (about 4.8 nm) and 0.25 mol/L (about 7.5 nm). Since it was anticipated that extremely thin/thick shell is adverse to the sensing properties, it is expected that the Fe₂O₃@ZnO with modest shell thickness would be beneficial to generating high sensitivity and selectivity. The assumption will be further confirmed in the following section.

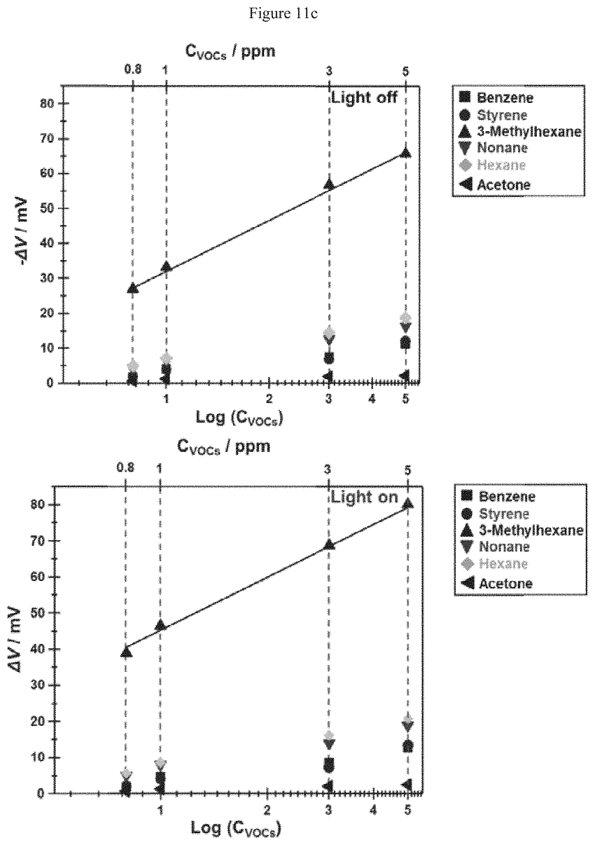

To confirm this assumption, sensing behavior of the YSZ-based sensors using Fe₂O₃—, ZnO— or Fe₂O₃@ZnO (with diverse shell thickness)-SE (vs. Mn-based RE) is evaluated. At the beginning stage, the fabrication temperature for the sensor and operational temperature are fixed at 900° C. and 425° C., note that these operating conditions are selected according to previous research experience¹³. FIG. 11(a) shows the response patterns for the electrochemical sensors (recorded at light off), depicted in the form of heat map in which different colors represent the corresponding sensing magnitude to a specific gas. As expected, the response behavior of the electrochemical sensors varied with the thickness of the ZnO shell. Apparently mutual interference is found the sensor solely use Fe₂O₃— or ZnO-SE (vs. Mn-based RE). Nevertheless, the coating of the photoactive ZnO brought about the obviously decrease in the response signal of benzene, styrene, nonane and hexane when the shell thickness is below 4.8 nm, while the response magnitude of the 3-methylhexane is slightly reduced, offering acceptable selectivity to 3-methylhexane for the electrochemical sensor using Fe₂O₃@ZnO (with the shell thickness of 4.8 nm)-SE vs. Mn-based RE. In contrast, a further increment of the shell thickness (≥7.5 nm) makes the sensing behavior more close to that of the sensor using ZnO-SE (vs. Mn-based RE) which is believed to be due to the blocked filter effect as discussed above.

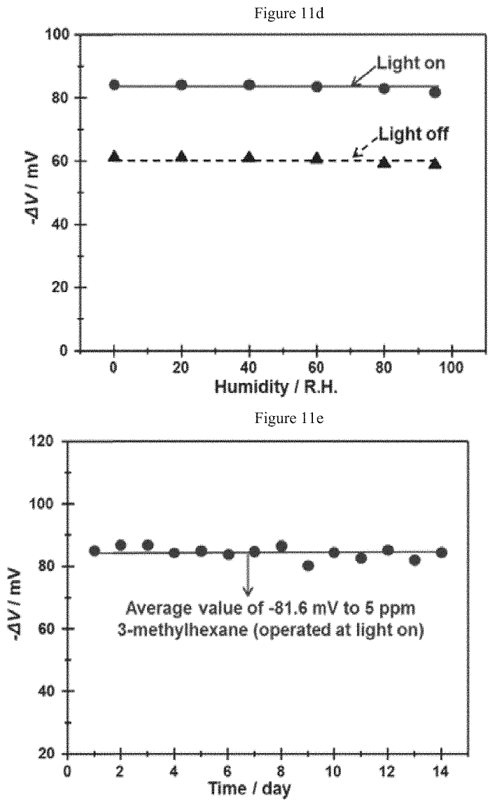

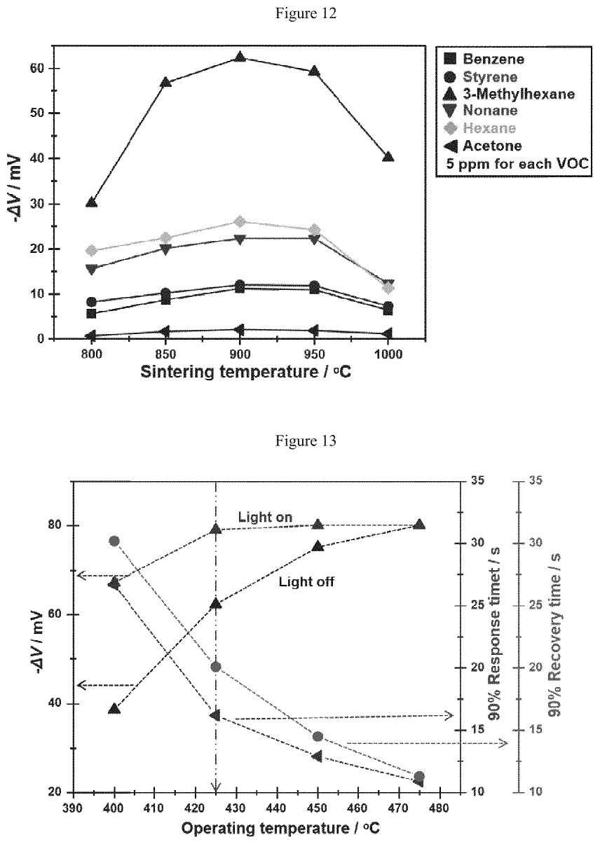

The fabricating and operating temperature of the sensor comprised of Fe₂O₃@ZnO (with the shell thickness of 4.8 nm)-SE vs. Mn-based RE is optimized and the relevant results are shown in FIGS. 12 and 13 of the Supporting Information. In summary, the sensor demonstrates optimal sensing properties (including the response/recovery rate) at the fabricating temperature of 900° C., with the 90% response/recovery time of 17 s and 21 s, respectively. Regarding the operational temperature, it was found that the sensor operated at 425° C. demonstrate maximum response signal to 5 ppm 3-methylhexane when been illuminated. Consequently, the fabricating/operating temperature of the sensor is fixed at 900° C./425° C. in this study. FIG. 11(b), (c) give the comparison of the sensing performance of the sensor using Fe₂O₃@ZnO (with the shell thickness of 4.8 nm)-SE vs. Mn-based RE, operated at light off or on. Interestingly, response signal of the sensor to 3-methylhexane is essentially enhanced, while its selectivity is maintained even been illuminated. The response signal recorded at light on (−81.3 mV to 5 ppm 3-methylhexane) is almost 1.3 times higher than that of the value obtained at light off (−64.2 mV). Moreover, the sensor demonstrates acceptable selectivity and linear relationship between the response signal (ΔV) and the logarithm of 3-methylhexane concentration, no matter operated at light off or on. The humidity effects on the sensing performance of the sensor is also investigated since the breath samples contain a massive amount of water vapor. Minor variation (within 3 mV) on the response magnitude of 5 ppm 3-methylhexane is observed in the water vapor range of 0 (dry)-95% (R.H.) (FIG. 11(d)). This is due to the water vapor trends to desorbed at such high operating temperature (425° C.), thus, water vapor can't occupy the reaction sites and block the electrochemical reaction. Long-term stability is another concerned issue for real clinic application, hence, the variation of the response magnitude of the sensor to 3-methylhexane (5 ppm) upon illumination is continuously examined for 2 weeks. It can be confirmed that acceptable response stability with the average response value of −81.6 mV is witnessed for the sensor even been operated at 425° C. for 14 days. Furthermore, results shown in table I suggest that the detection limit of the sensor to 3-methylhexane can be even extended to 0.072 ppm when upon illumination which is helpful to sense the changes of 3-methylhexane level in breath samples. Conclusively, the light-regulated electrochemical reaction assisted core-shell heterostructure (with tailor-made shell thickness) could indeed enhance the sensitivity, selectivity and the detection limit, paving the new way of designing future smart sensing devices for volatile markers surveillance.

TABLE 1
Sensing magnitude at 0.8 ppm, sensitivity and detection limit for the sensor
using Fe₂O₃@ZnO (with shell thickness of 4.8 nm)-SE vs. Mn-based RE,
operated at light off and light on, toward 6 kinds of volatile markers.

| | | −ΔV (at 0.8 ppm)/mV | | Sensitivity/(mV/Dec.) | | Detection limit/ppm | |
|---|---|---|---|---|---|---|---|
| Material | VOCs' | Light off | Light on | Light off | Light on | Light off | Light on |
| Fe₂O₃@ZnO | Benzene | 1.5 | 1.9 | 10.7 | 12.1 | 0.768 | 0.701 |
| (with shell | Styrene | 2.1 | 2.3 | 11.1 | 12.5 | 0.687 | 0.577 |
| thickness of | 3-Methylhexane | 27.2 | 38.9 | 48.5 | 50.2 | 0.127 | 0.072 |
| 4.8 nm) | Nonane | 4.1 | 5.1 | 14.4 | 16.1 | 0.589 | 0.493 |
| | Hexane | 5.0 | 5.7 | 16.8 | 18.0 | 0.521 | 0.494 |
| | Acetone | 0.5 | 0.7 | 1.73 | 2.07 | 0.972 | 0.968 |

For the purpose of achieving high performance in volatile markers surveillance, the strategy of designing light-regulated electrochemical reaction assisted core-shell heterostructure is proposed. Impact of the core species, shell thickness and illumination on the response behavior of the electrochemical sensor that using the core-shell sensing materials (as the SE) is thoroughly studied. Typically, among various core candidates. Fe₂O₃ was able to selectively remove most of the volatile markers (e.g. benzene, styrene, nonane, hexane and acetone) except the 3-methylhexane. Based on the finding, an electrochemical sensor that using Fe₂O@ZnO-SE vs. Mn-based RE is fabricated and its sensing performance is investigated. It is found that core-shell Fe₂O₃ZnO with the shell thickness of 4.8 nm offers the electrochemical sensor acceptable selectivity to 3-methylhexane. Particularly, sensing properties of the sensor are greatly enhanced upon illumination. In conclusion, benefiting from the simultaneously enhanced sensitivity and selectivity; it is anticipated that the strategy proposed in the research will be a starting point for the design of smarter sensing devices. Additionally, it should be particularly noted that since the filter effect to specific gases can be manipulated by replacing the Fe₂O₃ with other catalytic active core candidates, the selectivity of the sensor is speculated to be artificially tailored which needs to put more efforts on catalytic chemistry in the future.

What is claimed is:

1. A sensing electrode for detecting at least one target gas in a gas mixture having at least one interference gas, said sensing electrode comprises:
a. a layer of sensing nanoparticles;
b. a reaction interface; and
c. a solid state electrolyte;
wherein each of said sensing nanoparticles comprises a catalytic core and a photoactive porous shell, said catalytic core breaks down said at least one interference gas, said photoactive porous shell enhances electrochemical reaction at said reaction interface when illuminated with light of a specific wavelength.

2. The sensing electrode of claim 1, wherein said photoactive porous shell has a thickness of 3 nm to 10 nm.

3. The sensing electrode of claim 1, wherein said catalytic core is a metal oxide or metallic nanoparticle.

4. The sensing electrode of claim 3, wherein said metal oxide or metallic nanoparticle is selected from the group consisting of Fe₂O₃, In₂O₃, Au, Ag and Nb₂O₅.

5. The sensing electrode of claim 1, wherein said photoactive porous shell is made of ZnO.

6. The sensing electrode of claim 1, wherein said photoactive porous shell is made of ZnO based materials.

7. The sensing electrode of claim 1, wherein said target gas comprises a 3-methyl-alkyl group.

8. The sensing electrode of claim 7, wherein said target gas is 3-methylhexane.

9. The sensing electrode of claim 1, said interference gas is selected from the group consisting of benzene, styrene, nonane, hexane, 3-methylhexane, 2-ethylhexanol, 3-methylhexane, 5-ethyl-3-methyloctane, acetone, ethanol, ethyl acetate, ethyl-benzene, isononane, isoprene, nonanal, styrene, toluene, and undecane.

10. The sensing electrode of claim 1, wherein said specific wavelength ranges from 380-840 nm.

11. The sensing electrode of claim 1, wherein said solid state electrolyte is an oxygen ion conductor.

12. The sensing electrode of claim 11, wherein said solid state electrolyte is yttria-stabilized zirconia.

13. The sensing electrode of claim 1, wherein said catalytic core breaks down said at least one interference gas at a temperature above 400° C.

14. A sensor comprising said sensing electrode of claim 1.

15. A method for detecting at least one target gas in a gas mixture having at least one interference gas using said sensing electrode of claim 1, comprising the steps of:
a. providing said sensing electrode and a reference electrode;
b. illuminating said sensing electrode with light of said specific wavelength;
c. providing said gas mixture to said sensing electrode; and
d. measuring electric potential difference between said sensing electrode and said reference electrode.

16. The method of claim 16, said step (c) is conducted at a temperature above 400° C.

17. The method of claim 16, said target gas is at a concentration of 0-100 ppm.

18. The method of claim 16, said interference gas is a concentration below 5 ppm.

19. The method of claim 16, said target gas comprises a 3-methyl-alkyl group.

20. The method of claim 16, said interference gas is selected from the group consisting of benzene, styrene, nonane, hexane, 3-methylhexane, 2-ethylhexanol, 3-methylhexane, 5-ethyl-3-methyloctane, acetone, ethanol, ethyl acetate, ethyl-benzene, isononane, isoprene, nonanal, styrene, toluene, and undecane.
